(19) United States
(12) Patent Application Publication
(10) Pub. No.: US 2022/0240799 A1
(43) Pub. Date: Aug. 4, 2022

(54) BLOOD FLOW ANALYSIS DEVICE AND BIOLOGICAL INFORMATION ANALYSIS SYSTEM

(71) Applicant: HITACHI, LTD., Tokyo (JP)

(72) Inventors: Seiji MURATA, Tokyo (JP); Takahiro MATSUDA, Tokyo (JP); Satoshi OUCHI, Tokyo (JP)

(21) Appl. No.: 17/575,884

(22) Filed: Jan. 14, 2022

(30) Foreign Application Priority Data
Feb. 4, 2021 (JP) 2021-016765

(51) Int. Cl. A61B 5/026; A61B 5/021

(52) U.S. Cl. CPC A61B 5/0261; A61B 5/02125; A61B 5/02116

(57) ABSTRACT

A blood flow analysis device according to the present invention detects blood flow distribution by using a visible light image, compares blood flows between different measurement regions by using the blood flow distribution, and analyzes a correlation between blood flows simultaneously generated in different measurement regions.

### CROSS-REFERENCE TO RELATED APPLICATION

The present application claims priority from Japanese application JP2021-016765, filed on Feb. 4, 2021, the contents of which is hereby incorporated by reference into this application.

### BACKGROUND OF THE INVENTION

### 1. Field of the Invention

The present invention relates to a device that analyzes blood flow distribution of a subject.

### 2. Description of the Related Art

There is a technique of extracting a minute change in blood color from a series of images obtained by photographing a biological tissue of a human to detect a state change related to blood flow. The present technique is implemented by detecting an optical characteristic of a living body that change depending on blood flow from a captured video.

An example of the above technique is, for example, JP 2016-077890 A. JP 2016-077890 A describes, with an object of “measuring blood pressure in a non-contact manner”, a technique of “including an image acquisition unit that acquires a skin image in which a skin of the user is captured, a pulse wave timing calculation unit that calculates a temporal change in luminance in the skin image using the skin image and calculates time information indicating a time at which luminance peaks in the temporal change in luminance as a pulse wave timing, a millimeter wave acquisition unit that acquires a signal of a radio wave reflected by the user that is received by a receiving antenna, a heartbeat timing calculation unit that calculates a temporal change in a distance between the user and the receiving antenna using the signal of the radio wave acquired by the millimeter wave acquisition unit and calculates time information indicating a time at which the distance peaks in the temporal change in the distance as a heartbeat timing, and a blood pressure determination unit that determines a blood pressure of the user on the basis of a time difference between a pulse wave timing and the heartbeat timing” (see Abstract).

### SUMMARY OF THE INVENTION

A current non-contact blood flow state detection technique can detect an amplitude and a timing of reaching a peak of a pulse wave in a specific location. The present technique is used for measuring biological information such as a heartbeat and a blood pressure in a non-contact manner instead of a contact type measuring device that has been conventionally used. At present, there is an increasing social need to monitor the physical and mental state such as fatigue and stress in more situations without imposing a burden on the person to be measured in order to self-check a health condition of a human and improve the quality of life and working environment. In the future, in order to meet these needs, the technique is expected to be used in a form of estimating another piece of biological information from a state of blood flow.

In order to meet the above needs, Problems (1) and (2) described below that occur need to be solved. In particular, in a situation of detecting a change in the state of a human without an action, it is not possible to visually determine the change in the state, and the problems become remarkable.

Problem (1): Acquire more pieces of information from a state of blood flow.

Problem (2): Shorten a measurement time.

For Problem (1), it is necessary to spatially increase an amount of information on blood flow. For Problem (2), an index that allows immediate analysis of a large amount of information is required.

JP 2016-077890 A discloses a method including the receiving antenna that detects a heartbeat timing using a millimeter wave reflected by the user because information of a pulse wave timing obtained from information from an acquired skin image is not enough. In the configuration of JP 2016-077890 A, it is necessary to include a plurality of types of sensors of the image acquisition unit and the receiving antenna, and in addition, only one type of blood flow among many types of biological information is obtained by the sensors. Further, in the literature, since a peak time of a pulse and a heartbeat is detected, information cannot be acquired without spending time that is inevitably longer than a cycle of the pulse and the heartbeat.

The present invention has been made in view of the above problems, and an object of the present invention is to provide a blood flow analysis device capable of estimating a human state at a high speed and obtaining various types of information without imposing a burden of measurement on a subject.

A blood flow analysis device according to the present invention detects blood flow distribution by using a visible light image, compares blood flows between different measurement regions by using the blood flow distribution, and analyzes a correlation between blood flows simultaneously generated in different measurement regions.

According to the blood flow analysis device according to the present invention, it is possible to estimate a human state at a high speed and obtain various types of information without imposing a burden of measurement on a subject.

### BRIEF DESCRIPTION OF THE DRAWINGS

FIG. 1 is a configuration diagram of a blood flow analysis device 1 according to a first embodiment;

FIG. 2 is a configuration diagram of a biological information analysis system 2 according to a second embodiment;

FIG. 3 is a diagram illustrating an experimental result of blood flow states in two measurement regions at rest;

FIG. 4 is a diagram illustrating a correlation between blood flow states in two measurement regions at rest corresponding to FIG. 3;

FIG. 5 is a diagram illustrating blood flow states in two measurement regions when a subject is at rest for up to 60 seconds and then is active;

FIG. 6 is a diagram illustrating a correlation between blood flow states in two measurement regions when a subject shifts from a resting state to an active state;

FIG. 7 is a diagram illustrating a correlation between blood flow states in two measurement regions when a subject is active;

FIG. 8 is a configuration diagram of the biological information analysis system 2 according to a third embodiment;

FIG. 9 is a diagram illustrating a measurement region in the third embodiment; and

FIG. 10 is a configuration diagram of the biological information analysis system 2 according to a fourth embodiment.

### DESCRIPTION OF THE PREFERRED EMBODIMENTS

### First Embodiment

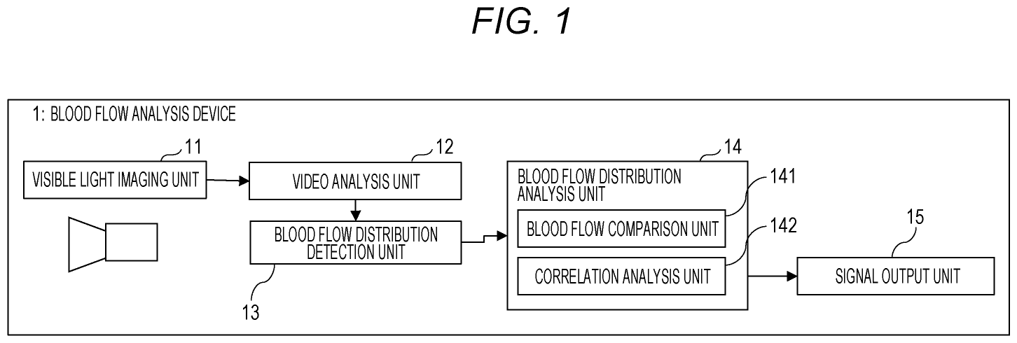

FIG. 1 is a configuration diagram of a blood flow analysis device 1 according to a first embodiment of the present invention. The blood flow analysis device 1 is a device that analyzes blood flow of a subject. The blood flow analysis device 1 includes a visible light imaging unit 11, a video analysis unit 12, a blood flow distribution detection unit 13, a blood flow distribution analysis unit 14, and a signal output unit 15. The blood flow distribution analysis unit 14 further includes a blood flow comparison unit 141 and a correlation analysis unit 142.

The visible light imaging unit 11 has a function of receiving visible light in a measurement region, converting a light intensity spatially distributed at least at two or more points into a signal, and outputting the signal. As a sensor that converts a light intensity into a signal, a CCD sensor or a CMOS sensor may be used. An optical system such as a lens or a mirror for forming an image of an object on a sensor may be provided. Furthermore, intensities of red light, green light, and blue light may be acquired for each unit space by using a color filter arranged corresponding to a pixel of the sensor. In particular, the color filter is desirably selected in accordance with an absorption spectrum of a pigment contained in blood. In the case of a human, a color filter having sensitivity in a red region where the absorbance of hemoglobin contained in blood is low is desirably included. As a red filter, for example, a filter that allows light having a wavelength in the vicinity of 600 nm to 700 nm to pass through can be used. Further, it is also possible to use a color filter that allows green light that reaches to capillaries existing under skin but does not easily penetrate deeper than the capillaries to pass through. As a green filter, for example, a filter that allows light having a wavelength of about 546 nm to pass through can be used. Further, a blue light through a filter that hardly permeates the inside of a living body can be used together. As the blue filter, for example, a filter that allows light having a wavelength in the vicinity of 436 nm to pass through can be used. A light receiving unit having sensitivity to infrared light may be provided, and a filter that allows only infrared light to pass through may be incorporated in a visible light camera. Further, the filter may be provided as an imaging unit separately from the visible light camera. As the infrared light filter, for example, a filter that allows light having a wavelength in the vicinity of 830 nm to pass through can be used. When infrared light is used, since it penetrates deep into a living body, blood flow in a blood vessel existing in a deep portion can be detected. Further, in a case where light in an infrared region is used, oxygen saturation concentration in blood can be evaluated. Hemoglobin has different absorbance depending on whether or not it is combined with oxygen, and for example, when light having a wavelength in the vicinity of 665 nm is used, a difference in optical characteristics is remarkably exhibited. On the other hand, in the infrared region, the absorbance is low and absorption is little regardless of the oxygen saturation concentration. Using this, it is possible to estimate oxygen saturation, which is a proportion of hemoglobin with which oxygen in the blood is combined, from a ratio of the absorbance in the infrared region and light in the vicinity of 665 nm.

The sensor that converts the light intensity into a signal included in the visible light imaging unit 11 is described assuming a sensor in which pixels are arranged in a two-dimensional manner. However, the configuration may be such that a photodetector that obtains the light intensity of one point as a signal and a plurality of optical systems that collect light from a measurement target are combined to obtain a measurement target located spatially separately. A color filter may be provided.

The video analysis unit 12 has a function of receiving a video signal from the visible light imaging unit 11, performing image analysis, and delivering the signal to the blood flow distribution detection unit 13. For example, only information of a necessary specific region in the received video signal is cut out, only video information necessary for processing is extracted, and unnecessary processing is omitted. Further, in a case where the vicinity of a pixel of a calculation target is sufficiently homogeneous, the resolution may be reduced to speed up the processing. As described above, unnecessary processing can be omitted by receiving a video signal and performing image processing.

The blood flow distribution detection unit 13 obtains information on blood flow distribution from the video signal transmitted from the video analysis unit 12. Specifically, the video signal is separated by color. For example, blood flow distribution can be obtained using a red signal (hereinafter referred to as an R signal). In a case where the measurement target of the blood flow distribution is a human living body, red light penetrates into the human living body and is partially absorbed, and other light is diffused and reflected. Among the light, light emitted from the living body can be captured by the visible light imaging unit 11. When the blood flow changes, the thickness of the blood vessel changes and a blood flow rate flowing inside changes, and an optical characteristic changes accordingly. Since this change in the optical characteristic also occurs in a red wavelength region reaching the blood vessel, a change in blood flow can be seen. Further, since the visible light imaging unit 11 can spatially acquire light intensity distribution, it is possible to detect blood flow distribution.

The blood flow distribution analysis unit 14 has a function of analyzing information on a state of blood flow from the blood flow distribution detected by the blood flow distribution detection unit 13. For example, a pulse that is biological information can be calculated. For example, a pulse can be detected in a manner that a change in blood flow with time is applied with Fourier transform and frequency analysis. Other than the above, by recording a time at which a pulse reaches the peak and recording a change in the pulse cycle with a sufficient length, the balance of an autonomic nerve can be estimated using the analysis.

The blood flow distribution analysis unit 14 includes a blood flow comparison unit 141 that compares information on a plurality of blood flows, and a correlation analysis unit 142 that analyzes a relationship between intensities of a plurality of blood flows at a certain time point.

The blood flow comparison unit 141 has a function of comparing changes in blood flow intensity measured in different locations. Even in a living body, likelihood of change in blood flow varies depending on a part. A ratio of the change in blood flow can be measured by comparing blood flow measured in the part where blood flow is likely to change and the part where blood flow is unlikely to change. As a phenomenon in which the state of blood flow is partially changed, there is a phenomenon caused by opening and closing of an arteriovenous anastomosis by the autonomic nerve. The blood flow comparison unit 141 can detect a change in balance of the autonomic nerve by measuring a partial change in the state of blood flow and estimating a change in the arteriovenous anastomosis spending time until the distribution of blood flow changes sufficiently.

The correlation analysis unit 142 has a function of taking a value with a state of blood flow in each region as an axis for the state of blood flow in a plurality of regions at a certain moment, and analyzing information regarding blood flow from the distribution. For example, it is assumed that there are a first measurement region and a second measurement region. In the graph taking two axes, when a signal corresponding to a blood flow rate in the first measurement region at a certain moment (time t) is B1 and a signal corresponding to a blood flow rate in the second measurement region is B2, a point taking coordinates (B1, B2) can be drawn. When the point is sampled within a certain time, distribution is obtained. The correlation analysis unit 142 has a function of calculating and outputting an index by analyzing a state of the distribution.

Examples of an evaluation item for evaluating a state of distribution include a center of gravity, a slope, an intercept, and a correlation coefficient. The center of gravity indicates a representative state of blood flow balance within measurement time. The slope depends on a ratio of amplitudes in different measurement targets. The intercept indicates an offset with respect to a blood flow rate. Focusing on a temporal change in the blood flow rate, processing of canceling an offset is also possible. The correlation coefficient depends on a phase of a fluctuation of the blood flow rate of a measurement target and a ratio of an amplitude of the blood flow rate. The correlation coefficient is high when the phases are aligned and the ratio of the amplitude does not change. As described above, since the amount determined by the phase and the ratio of the amplitude can be analyzed each time, sampling can be performed a plurality of times within a pulsating cycle of blood flow. With the above-described function of the correlation analysis unit 142, it is possible to provide a means for analyzing and evaluating blood flow distribution at a higher speed than a pulse cycle.

The signal output unit 15 has a function of transmitting a signal as an output of at least one result (evaluation index of blood flow) calculated by the blood flow distribution analysis unit 14. For example, examples of the result output by the blood flow distribution analysis unit 14 include a heart rate, a correlation coefficient of the blood flow rates of the first measurement target and the second measurement target, and the like. The signal output unit 15 can also present these outputs by transmitting data to, for example, a device for visual presentation such as a monitor or a printer, or a speaker that performs notification by voice.

### First Embodiment: Conclusion

The blood flow analysis device 1 according to the present embodiment measures blood flow distribution from a video acquired in a non-contact manner, compares blood flows in different measurement regions using the blood flow distribution, and analyzes a correlation between blood flows simultaneously generated in different measurement regions. This makes it possible to measure blood flow in a short time and to obtain various types of information regarding the blood flow.

The blood flow analysis device 1 according to the present embodiment detects a state change in blood flow distribution in a time shorter than a pulse cycle. In this manner, various types of information other than a pulse regarding blood flow can be obtained within a time shorter than the pulse.

### Second Embodiment

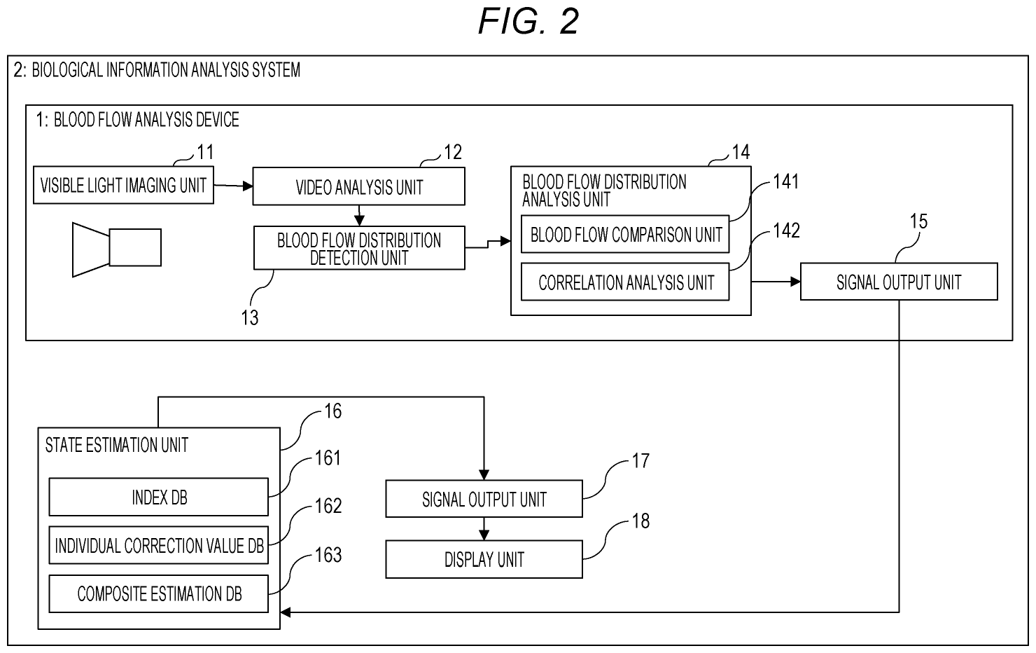

FIG. 2 is a configuration diagram of a biological information analysis system 2 according to a second embodiment of the present invention. The biological information analysis system 2 is a system that estimates a state of a subject using information on blood flow distribution analyzed by the blood flow analysis device 1. The biological information analysis system 2 includes a state estimation unit 16, a signal output unit 17, and a display unit 18 in addition to the blood flow analysis device 1. A result output from the signal output unit 15 is transmitted to the state estimation unit 16.

The state estimation unit 16 includes at least one databases for storing an index for determining a result received from the signal output unit 15. At least information on a type of evaluation and a boundary of determination is recorded in the database. In a case where the number pieces of information is small, an update frequency is low, and management is not required, a simply recorded data table may be used. Here, an example in which an index DB 161, an individual correction value DB 162, and a composite estimation DB 163 are provided is illustrated.

The index DB 161 stores a combination of an evaluation index, information on a boundary of determination, and a state of a subject. The state estimation unit 16 refers to the index DB 161 by using an evaluation index received from the signal output unit 15 to acquire a state of a subject corresponding to the evaluation index from the index DB 161. In this manner, a state of a subject can be estimated.

Even if the evaluation index of blood flow distribution is the same, a state of the living body may vary depending on an individual. The individual correction value DB 162 holds an individual correction value for correcting this individual difference. The state estimation unit 16 can estimate a state of a subject using the index DB 161 after correcting an individual difference by using the individual correction value DB 162.

The composite estimation DB 163 holds a relationship between a plurality of index values and a state of a subject. The state estimation unit 16 inputs a correlation coefficient of blood flow distribution and a pulse in a plurality of target portions to the composite estimation DB 163, for example, and obtains a state of a subject corresponding to these. For example, the composite estimation DB 163 holds an estimation result that a subject is resting calmly if the correlation coefficient is high and the pulse is low. In order to prevent the capacity required for recording from increasing by a combination of an index value and an estimation result, a combination of a related parameter, a correction coefficient, and a coefficient of a function for approximation may be held.

The signal output unit 17 has a function of transmitting a signal as an output of a result of estimating at least one or more human states. For example, the result output by the state estimation unit 16 includes a resting state and the like. The display unit 18 receives the estimation result and displays a signal so that the user can view the signal.

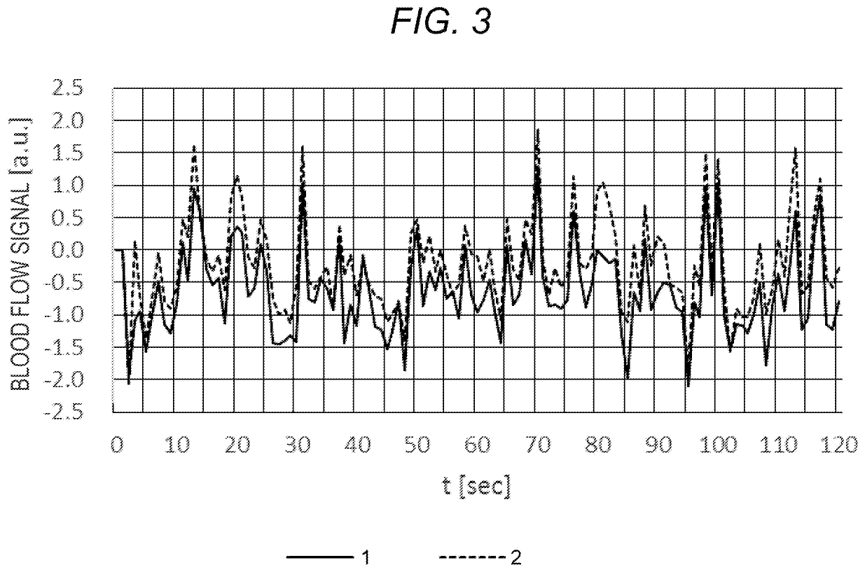

FIG. 3 is a diagram illustrating an experimental result of blood flow states in two measurement regions at rest. FIG. 3 illustrates a change in a blood flow state in the first measurement region and the second measurement region as measurement targets for blood flow, which are measured when a subject is at rest for 120 seconds. The horizontal axis represents time, and the vertical axis represents a signal value indicating increase or decrease of blood flow. In this experiment, the measurement regions are set so as to include the nose as the first measurement region and include a cheek as the second measurement region.

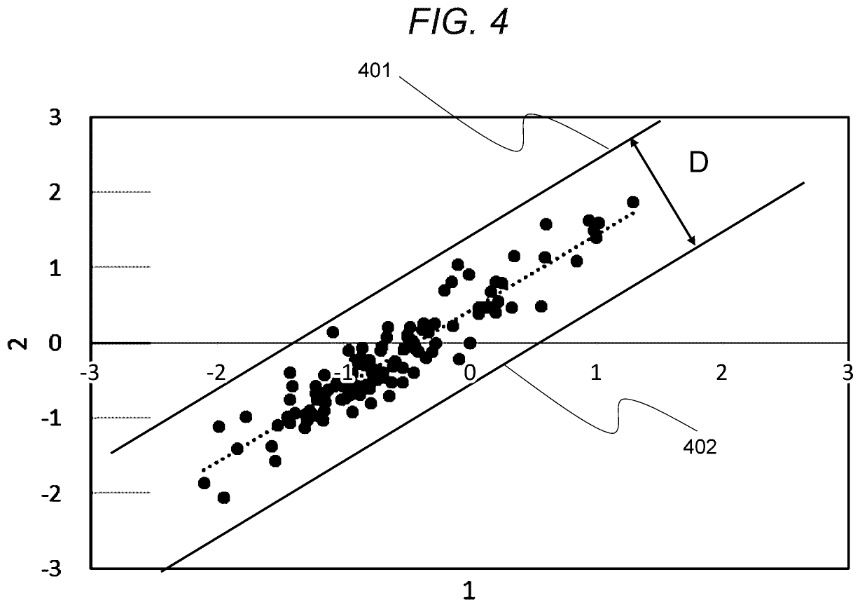

FIG. 4 is a diagram illustrating a correlation between blood flow states in two measurement regions at rest corresponding to FIG. 3. By taking a signal value of the blood flow state in the first measurement region at a certain moment on the horizontal axis and a signal value of the blood flow state in the second measurement region on the vertical axis, a state of blood flow in two measurement regions at a certain moment can be expressed by one point. FIG. 4 is obtained by drawing the above by using a measured value at rest for 120 seconds. These pieces of data can be evaluated by an approximate straight line derived from a plurality of measurement points. The approximate straight line includes a slope and an offset (dotted line in FIG. 4). As another evaluation method, the evaluation can be performed by the number of measurement points included in a region sandwiched between an upper limit 401 and a lower limit 402 separated by a certain distance D with the approximate straight line interposed between them, or a ratio of the measurement points to all the measurement points. Further, it is also possible to perform evaluation using a correlation coefficient or a variance of measurement data.

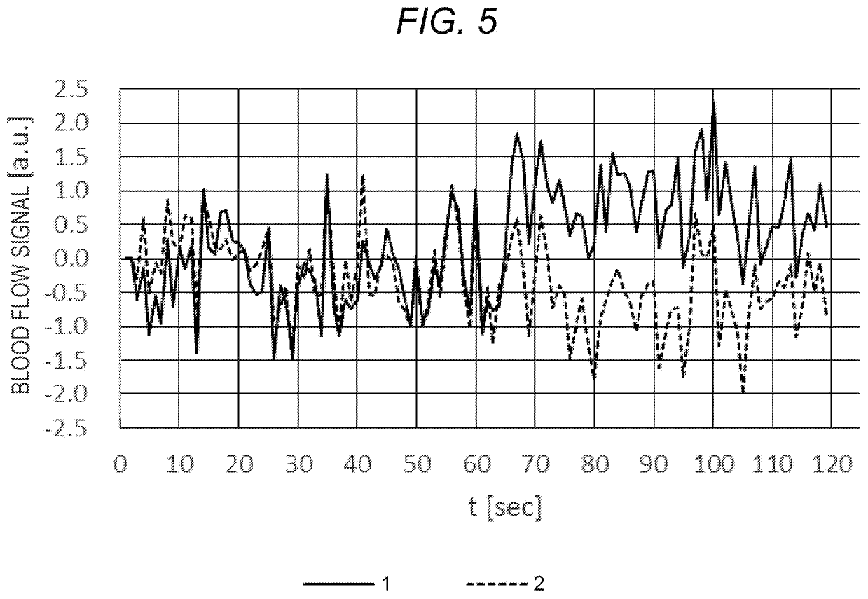

FIG. 5 is a diagram illustrating blood flow states in two measurement regions when a subject is at rest for up to 60 seconds and then is active. The measurement regions are under the same conditions as those in FIG. 3, and the regions are set so as to include the nose in the first measurement region and a cheek in the second measurement region. As an activity, mental calculation of adding two-digit numbers (that is, an activity without physical exercise) was performed. From around 65 seconds, it can be seen that two blood flow change tendencies are significantly deviated.

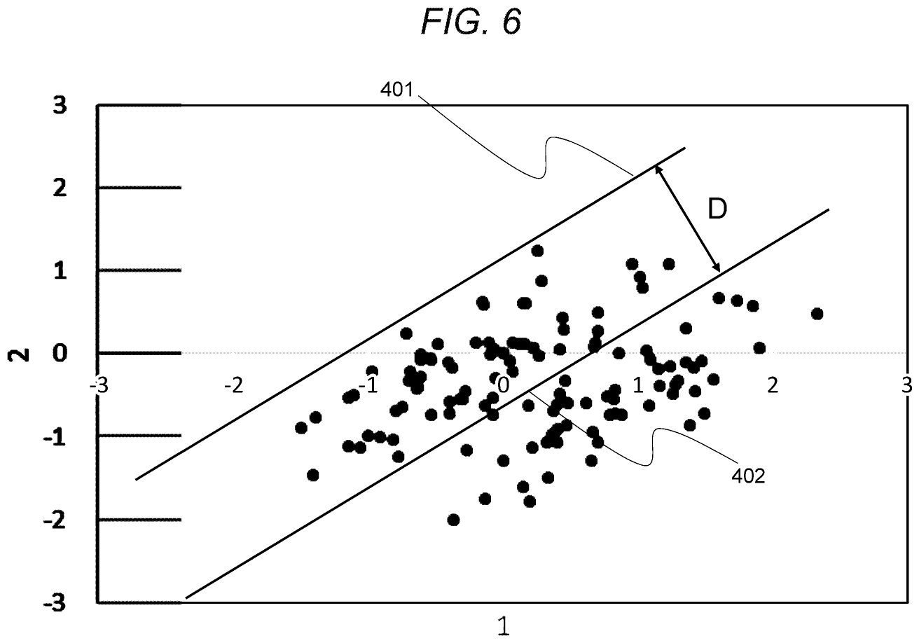

FIG. 6 is a diagram illustrating a correlation between blood flow states in two measurement regions when a subject shifts from a resting state to an active state. It can be seen that the dispersion of the measurement data is large unlike at rest. For reference, the upper limit 401 and the lower limit 402 illustrated in FIG. 4 are illustrated in an overlapping manner. It can be seen that there are many pieces of measurement data that are greatly deviated. The correlation coefficient is as low as 0.33. In particular, since the measurement data is the measurement data in which the resting time and the activity time are mixed, it can be seen that it is difficult to take a correlation.

The evaluation of a blood flow state is also related to an elapsed time required for shift of a state of a subject. For example, it is assumed that an occurring phenomenon is different between a case where the state changes suddenly and a case where the state changes gently. In view of the above, a temporal change rate of the state can also be used as an evaluation index. For example, in a case where the correlation of the measurement data suddenly collapses, it is considered that the state of the subject changes, and in a case where there is no change in the correlation, it is considered that the subject is in a stable state. Based on abnormality, for example, the state estimation unit 16 can estimate that the state of the subject is changed when the temporal change rate of the index value is equal to or more than a threshold, and can estimate that the state of the subject is stable when the temporal change rate is less than the threshold.

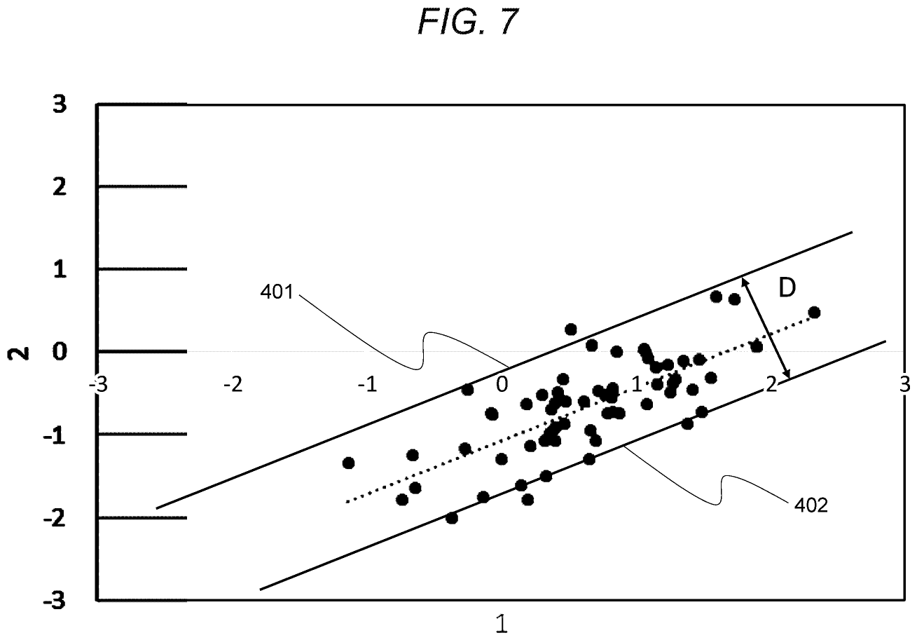

FIG. 7 is a diagram illustrating a correlation between blood flow states in two measurement regions when a subject is active. That is, the measurement data is extracted only at the time of activity. It can be seen that the dispersion of the measurement data is large. The correlation coefficient is 0.74.

Focusing on the correlation coefficients of the experiments illustrated in FIGS. 3 to 7, the correlation coefficient is as high as 0.94 at rest, as low as 0.33 during the shift from rest to activity, and as low as 0.74 at the time of activity. Although not illustrated, the correlation coefficient at rest that was measured at another opportunity was 0.81. From the above, a threshold of the correlation coefficient for determining rest and activity is preferably set between 0.74 and 0.81, and in a case where the correlation coefficient is lower than the threshold, it possibly indicates during the shift. At the time of the shift, it is desirable to refer to a correlation coefficient and a state change before and after the shift. The state estimation using the correlation coefficient or the evaluation index may be performed by the state estimation unit 16 or may be performed by the correlation analysis unit 142.

The relationship between the correlation coefficient and the motion state of the subject illustrated in FIGS. 3 to 7 particularly appears well between a measurement region where a blood flow change due to arteriovenous anastomosis is large and a measurement region where the blood flow change due to arteriovenous anastomosis is small. Therefore, in a case where the motion state of the subject is estimated using these correlation coefficients, it is desirable to use a correlation coefficient between such measurement regions. The nose and cheek are one example.

### Second Embodiment: Conclusion

The biological information analysis system 2 according to the second embodiment calculates an index value representing a correlation between an increase or decrease in blood flow in the first measurement region and an increase or decrease in blood flow in the second measurement region, and estimates a state of a subject according to the index value. This makes it possible to obtain information on a state of the subject that is difficult to obtain from blood flow distribution of only one location.

The biological information analysis system 2 according to the second embodiment (a) estimates that a subject is in a resting state when an index value indicating a correlation between an increase or decrease in blood flow in the first measurement region and an increase or decrease in the blood flow in the second measurement region is high (about 0.94 in the above example), (b) estimates that the subject is in an active state when the index value is slightly low (0.74 to 0.81 in the above example), and (c) estimates that the subject shifts between the resting state and the active state when the index value is low (0.33 in the above example). In this manner, the state of the subject can be accurately estimated.

### Third Embodiment

In a third embodiment of the present invention, a configuration example of reducing an analysis load of blood flow distribution by selecting only a range necessary for analysis of blood flow distribution in a subject image acquired in a non-contact manner.

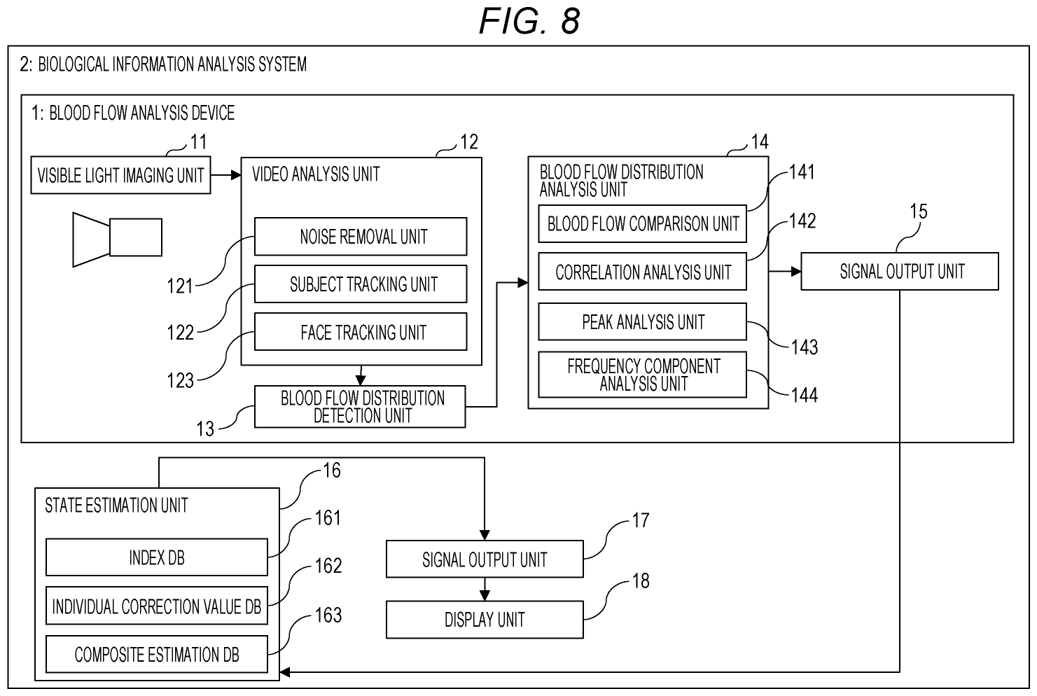

FIG. 8 is a configuration diagram of the biological information analysis system 2 according to the third embodiment. In addition to the configuration described in the second embodiment, the biological information analysis system 2 according to the third embodiment includes a noise removal unit 121, a subject tracking unit 122, and a face tracking unit 123 as submodules of the video analysis unit 12, and further includes a peak analysis unit 143 and a frequency component analysis unit 144 as submodules of the blood flow distribution analysis unit 14.

The noise removal unit 121 has a function of processing a video acquired by the video analysis unit 12 to remove unnecessary information. As a specific method of removing unnecessary information, a method of obtaining an average between a plurality of consecutive pixels and performing smoothing by using the average as a representative value of signal values of the pixels to remove noise can be used. According to the present method, it is possible to smooth a signal error randomly added for each pixel, such as one caused by noise of an electrical characteristic of a sensor included in the visible light imaging unit 11, and to obtain a signal intensity that can be treated as a representative value of a region near the pixel. By the noise removal of the present method, it is possible to obtain a video in which an amplitude of spatial noise is suppressed.

As another noise removal method, a method of removing noise by selecting a signal intensity having a highest appearance frequency as a representative value by using a histogram of signal intensities of a plurality of pixels can be used. In the present method, since a signal intensity that appears the most in the vicinity is selected, after noise is removed, noise in which the same value continue is reduced and a change is made small, and homogenized signal distribution is obtained. Since the method is a system of obtaining a value having a highest appearance frequency in a region in the vicinity of a measurement target, in a state where an error varies greatly, a situation where the numbers of pixels of the signal intensity that appears most frequently become equal and it is difficult to uniquely determine a signal intensity may occur. Therefore, it is desirable to apply the present method to an image after obtaining an average between pixels and removing noise.

As another noise removal method, a method of adjusting the balance with respect to a signal intensity for each color of the video acquired by the visible light imaging unit 11 can be employed. For example, a case of using a camera capable of obtaining light intensity distribution of three colors of red (R), green (G), and blue (B) generally used will be described. A method in which, when light intensity distribution in R, G, and B is obtained in a region as a measurement target, a highest intensity and a lowest intensity of each color are aligned, and an intermediate intensity is complemented, is employed, so that intensity ranges in which signal distribution of each color exists can be aligned. A highest intensity and a lowest intensity after the alignment may be scaled according to an upper limit value and a lower limit value of a level that a signal may take. For example, it is a method of distributing intensities in a range of 0% to 100% when handled in percentage, and in a range of 0 to 255 when handled in eight bits. According to the present method, it is possible to cancel a difference in sensitivity between colors determined by the visible light imaging unit 11 and obtain signal distribution for equally handling the colors.

As another method of removing noise, a method of adjusting the balance with respect to a signal intensity for each color from signal distribution of each color of a certain region in the video acquired by the visible light imaging unit 11 can be taken. For example, when light intensity distribution of R, G, and B is obtained, a characteristic amount representing light intensity distributions of each color in the region is obtained, and a signal of each color is corrected so as to align the characteristic amounts. Examples of the characteristic amount include a highest intensity, a lowest intensity, and a most frequent intensity. Examples of a coefficient necessary for the correction include an offset that means a difference in signal distribution, a gain indicating an intensity ratio of signal distribution, and the like. As a method of correcting in more detail, it is also possible to use an optional function in which a corrected signal value is associated with each color and each luminance, and adjustment using what is called a tone curve may be used. According to the present method, it is possible to perform correction in consideration of distribution of signal intensities of an intermediate color when signal intensities are aligned between colors.

In order to correct a signal intensity of each color, a method of measuring an inanimate object and determining a correction coefficient may be employed. In the present method, a video of an inanimate object captured by the visible light imaging unit 11 does not change due to a biological reaction, but is affected by a change in an optical environment, for example, a change in color and intensity of ambient light of uniform illumination. On the other hand, a video of a living body as a measurement target is affected not only by a change in a signal intensity due to a change in an optical characteristic due to a biological reaction but also by a change in color and intensity of ambient light. That is, by obtaining a correction coefficient of a signal intensity between each color by using a video of an inanimate object at a certain reference time and using a correction coefficient of a signal intensity between each color using a video of the inanimate object at another time of measurement, it is possible to offset an optical environmental change and reduce the influence in accordance with a condition at the certain reference time. This makes it possible to obtain a change due to an optical characteristic of a living body with high sensitivity.

In order to reduce a bias of intensity signals between colors and handle them equally at the time of photographing, an achromatic color in which the hue is suppressed is desirable for an inanimate object as a measurement target used for correcting a signal intensity of each color. Using an achromatic color having no hue a reference is useful to determine in which direction a color is biased. Further, in order to prevent a reflection peak of illumination light from occurring spatially, it is desirable to use a material having high diffusivity. For example, the diffusivity can be determined by confirming that it is difficult to specify a shape of a light emitting surface when lighting equipment having a finite light emitting surface is reflected on a measurement target. Since information necessary for obtaining a conversion function (tone curve) of a signal intensity between colors is obtained for each intermediate signal intensity, a gray scale inanimate object having intensity distribution is desirable.

The subject tracking unit 122 has an image tracking function of identifying where a subject appears in a video when the subject captured by the visible light imaging unit 11 moves in the video. As a method of image tracking a subject, an object tracking algorithm can be used. The object tracking algorithm is a function of recording data on appearance of an object as a measurement object and determining whether there is a corresponding object in a video captured by the visible light imaging unit 11. Not only the presence or absence of an object in a video but also a position and size of an object in the video is desirably identified.

As a method of achieving object tracking, a machine learning mechanism in which data related to an object is given in advance and learned, and the presence/absence, position, and size of the object are returned using an evaluation function obtained by learning may be used. According to the present method, even if an imaging target moves or an imaging direction of the visible light imaging unit 11 deviates, the position of a measurement target in a captured video can be identified, so that stable measurement can be performed.

As another method of object tracking, there is an image tracking method using optical flow. The optical flow is a method of obtaining a moving direction and a distance for each pixel or region that is considered to be most consistent by comparing and evaluating a video generated on the assumption that a certain pixel or region at a reference time moves in a certain direction from a video at the reference time when assuming that the pixel or region moves in the certain and by a certain distance when a certain time elapses and a video actually obtained after the certain time elapses. By using the present method, there is a possibility that tracking can be performed even if an imaging target is not learned in advance, and particularly when an optional imaging scene is targeted, it is possible to improve stability against movement, for example, shaking, of the visible light imaging unit 11.

The face tracking unit 123 has a function of tracking a face present in the video captured by the visible light imaging unit 11 and identifying a region in the captured video. By tracking a face portion, it is possible to effectively limit a measurement target region and to realize efficient processing by omitting unnecessary video processing. In addition to tracking the face, each part in the face (examples: eye, nose, and the like) may be tracked.

The face tracking is a function of learning a video of a plurality of faces and identifying which region in the captured video a face of a person is located by using an evaluation function obtained by the learning. The function may be configured using an evaluation function obtained using machine learning or deep learning. By performing the processing limitedly to a video region including a video of a face region identified by the present function, the processing of a region unnecessary for the measurement is avoided, and the efficient processing can be performed.

A position of a face part may be used as a reference of the measurement target region. The region in a video in which a part located on the face, for example, a specific part such as the forehead, an eyelid, an eye, the nose, a cheek, the upper lip, the lower lip, and an ear is included may be identified, and the position and size of the region may be used as a reference. For example, in order to set a measurement region between the eyebrows, a method in which after the positions of the eyes are identified, a region located between them is calculated and determined can be taken. With such a method, even if there is a part that cannot be directly identified, it is possible to define a measurement region from a relative position with reference to another identifiable part, and it is possible to improve the degree of freedom of measurement.

A function of holding data related to a face of an individual, determining whether a photographed person is a measurement target by performing collation by face tracking, and further narrowing down a measurement region to improve efficiency may be included. Furthermore, it is convenient to have a function of registering data regarding a face of an individual.

The peak analysis unit 143 captures a peak of a blood flow state, records and analyzes the time and the magnitude of the peak. The frequency component analysis unit 144 performs frequency analysis of a pulse in a change in a blood flow state. With these units, it is possible to estimate a human state by analyzing pulsation of a sufficiently large number of pulses. For example, an analysis result of these can be used to evaluate the balance of the autonomic nerve.

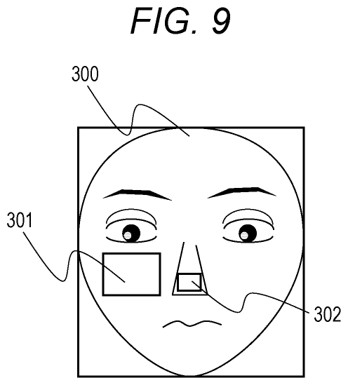

FIG. 9 is a diagram illustrating a measurement region in the third embodiment. A face region 300 identified by the face tracking is identified, a measurement region 301 is identified as a cheek by identification of a face part, and a measurement region 302 is identified as the nose. For example, the measurement region 301 can be used as the first measurement region, and the measurement region 302 can be used as the second measurement region.

### Third Embodiment: Conclusion

In the biological information analysis system 2 according to the third embodiment, the subject tracking unit 122 tracks a subject or the face tracking unit 123 tracks a face portion of a subject, so that it is possible to limit video information necessary for the blood flow distribution analysis. In this manner, the load of analyzing the blood flow distribution can be reduced, so that it is possible to detect a state change in the blood flow distribution in a short time.

### Fourth Embodiment

As a fourth embodiment of the present invention, a configuration example of estimating a human state in more detail based on a plurality of different pieces of data using learning data received from outside a blood flow analysis device 100.

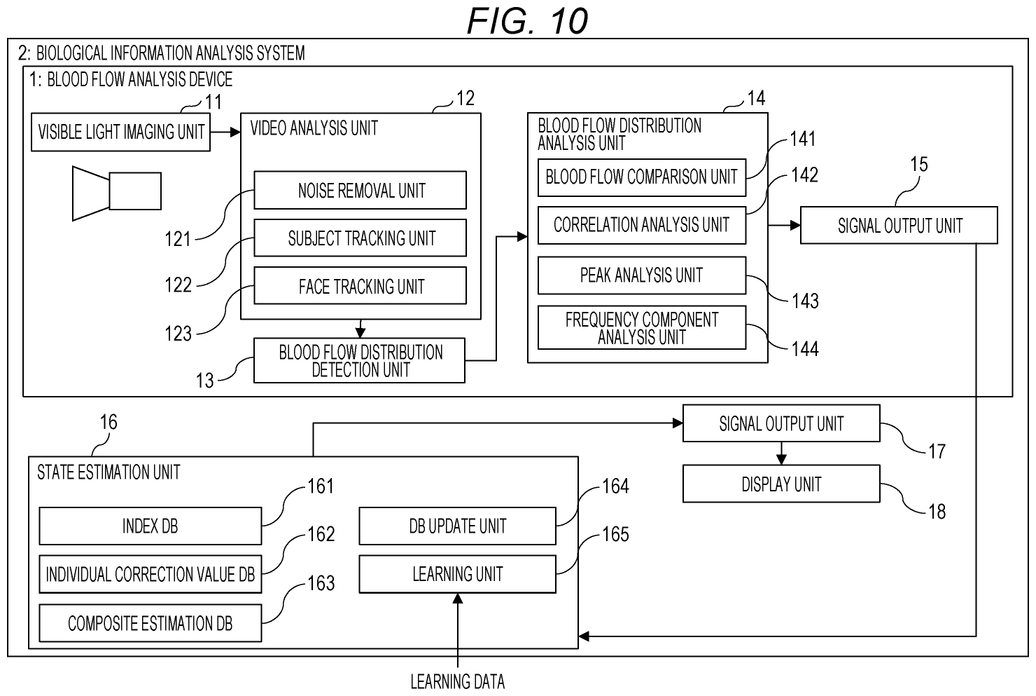

FIG. 10 is a configuration diagram of the biological information analysis system 2 according to the fourth embodiment. In the fourth embodiment, the state estimation unit 16 includes a DB update unit 164 and a learning unit 165 in addition to the configuration described in the second to third embodiments. The DB update unit 164 takes in learning data provided from the outside and updates the content of various databases included in the state estimation unit 16. The learning unit 165 performs machine learning or the like using the learning data to obtain an evaluation function necessary for state estimation.

The DB update unit 164 has a function of updating the content of the index DB 161, the individual correction value DB 162, and the composite estimation DB 163. The DB update unit 164 identifies and updates a database to be updated and an item and content to be updated according to a given command. It is desirable to have a determination function of not executing a command so as not to generate an error in view of consistency with another database in a case where there is an inconsistency in the command.

The learning unit 165 has a function of updating an evaluation function necessary for state estimation using data from the signal output unit 15 of the blood flow analysis device 100 or learning data obtained by other means. For example, when the input data is a measurement result by the blood flow analysis device 100, a measurement condition, and human state confirmation data indicating an actually clarified human state, it is possible to employ a configuration in which the measurement result, the measurement condition, and the human state confirmation data are made to correspond to each other, and an evaluation function is learned using them as teacher data. With this configuration, the probability of correctly estimating a human state can be improved using an evaluation function generated by learning even under a condition that has never been measured before.

For example, by causing machine learning to be performed using the data from the signal output unit 15 of the blood flow analysis device 100 and a state of the a photographed person obtained by corresponding other means as teaching data, a learning model can be constructed using supervised learning for estimating a state of the photographed person from the output of the signal output unit 15 of the blood flow analysis device 100. As another means, it is possible to improve the accuracy of estimating a state by using a data set in which data regarding a motion of a photographed person is added to the teacher data.

Further, in addition to the data from the signal output unit 15 of the blood flow analysis device 100 and the data set of a state of a photographed person obtained by corresponding other means, a model for estimating the state of the photographed person may be constructed by utilizing deep learning using time series information such as order and time of the measurement as learning data. For example, a recurrent neural network (RNN) can be used as a learning method. In particular, since a model for estimating a state of a photographed person can be constructed in consideration of information related to an increase and decrease in a change of blood flow and a change rate, the accuracy can be improved while the number of types of states to be estimated is increased.

The DB update unit 164 may be configured to reinforce learning by inserting an item of a new condition into an existing condition used for learning by the learning unit 165. According to this configuration, even in a case where the degree of freedom of a condition is low and a change for correctly determining an evaluation function is not made, it is possible to give room for improving the probability of correct determination by increasing the number of conditions. The learning data can target not only the index DB 161 but also the individual correction value DB 162 and the composite estimation DB 163.

When learning is performed using data of the individual correction value DB 162, an evaluation function considering an individual difference can be obtained. When a new person to be measured is added, a method in which, from a result of simple measurement, an already-registered individual correction value showing a characteristic similar to that of the person to be measured is given as an initial value with respect to an individual correction value, and then learning is performed based on a measurement result with a changed condition and a state of a person, so that convergence occurs faster, can be taken.

When learning is performed using data included in the composite estimation DB 163, more detailed data can be obtained from learning data obtained from the outside and a plurality of types of data obtained from the blood flow analysis device 100. For example, when a certain subject is measured, if a correlation value of blood flow output by the correlation analysis unit 142 is as high as 0.97 and is stable for one minute, it can be seen that there is no change in the state and the state is stable. When a measurement result of a pulse is 60 bpm and a normal pulse of the subject included in the individual correction value DB 162 is 64 bpm, it can be determined that the pulse is lower than the normal pulse. Since it is generally known that a person is relaxed when a pulse is low, it is possible to estimate a more detailed state that the subject is relaxed and stable. When a signal indicating that a body is not moved is given as a result of photographing with another camera as learning data, it is estimated that the subject is resting, and it is possible to perform determination with higher accuracy that the subject is stable in a relaxed state. As described above, a human state can be estimated in more detail by combining different pieces of information.

It is also possible to use a plurality of types of learning data, and in a case where the types are wide-ranging, a method of lowering the number of dimensions during calculation to increase calculation efficiency using a deep learning model may be used. For example, in a case where data from the signal output unit 15 of the blood flow analysis device 100 and an appearance obtained by measuring blood flow of a corresponding photographed person are combined to form a learning data set, a data configuration in which the number of dimensions of data increases by the spatial resolution of the appearance may be taken. The method is effective for increasing calculation efficiency in a case where the number of dimensions of learning data is dramatically increased as described above. As a measurement example assuming this condition, a case in which a component of a video having information of an expression or deformation based on contraction of a muscle obtained from an appearance is used in combination with data regarding blood flow for learning data can be considered. Under such a condition, since the number of dimensions of learning data increases, a learning model that can cope with many conditions can be realized, types of states of a photographed person that can be estimated are expanded, and stability and accuracy can be improved.

### Fourth Embodiment: Conclusion

In the biological information analysis system 2 according to the fourth embodiment, the learning unit 165 learns a relationship between a motion state of a subject and a measurement result using learning data, so that estimation accuracy of the motion state can be continuously improved. Further, by using data acquired from a device other than the blood flow analysis device 1 as learning data, it is possible to estimate a human state in more detail based on a plurality of different pieces of data.

### Variation of Present Invention

The present invention is not limited to the above embodiments and includes a variety of variations. For example, the above embodiments are described in detail for easy understanding of the present invention, and the present invention is not necessarily limited to embodiments that include all the described configurations. Part of a configuration of a certain embodiment can be replaced with a configuration of another embodiment, and a configuration of a certain embodiment can be added to a configuration of another embodiment. Further, for part of a configuration of each embodiment, other configurations may be added, removed, or replaced with.

In the above embodiments, the video analysis unit 12, the blood flow distribution analysis unit 14, the blood flow distribution detection unit 13, the signal output unit 15, the state estimation unit 16, the signal output unit 17, and the display unit 18 can be configured by hardware such as a circuit device in which functions of the above units are mounted, or can be configured by an arithmetic device executing software in which functions of the above units are mounted.

In the above embodiments, the state estimation unit 16 can also be a constituent of the blood flow analysis device 1. In that case, the state estimation unit 16 can be configured as an independent functional unit or can be configured as a part of another functional unit. For example, the state estimation unit 16 may be configured as a part of the blood flow distribution analysis unit 14, and the blood flow distribution analysis unit 14 may estimate a state of a subject and output a result of the estimation.

What is claimed is:

1. A blood flow analysis device for analyzing blood flow of a subject, the blood flow analysis device comprising:
a visible light photographing unit that images light distribution of visible light in a measurement region for measuring the blood flow;
a blood flow distribution detection unit that detects distribution of the blood flow by using a visible light image captured by the visible light photographing unit;
a blood flow comparison unit that compares blood flows between different measurement regions by using blood flow distribution detected by the blood flow distribution detection unit; and
a correlation analysis unit that analyzes a correlation between blood flows simultaneously generated in different measurement regions by using blood flow distribution detected by the blood flow distribution detection unit.

2. The blood flow analysis device according to claim 1, wherein
the correlation analysis unit calculates an amount represented by a phase and an amplitude of each of blood flows simultaneously generated in different measurement regions at a frequency higher than a pulse cycle the subject so as to analyze the correlation at a frequency higher than the pulse cycle.

3. The blood flow analysis device according to claim 1, wherein
the correlation analysis unit calculates an index value representing a correlation between an increase or decrease in a first blood flow in a first measurement region and an increase or decrease in a second blood flow simultaneously generated with the first blood flow in a second measurement region different from the first measurement region, and
the correlation analysis unit estimates a motion performed by the subject according to the index value and outputs a result thereof.

4. The blood flow analysis device according to claim 3, wherein
the correlation analysis unit outputs an estimation result indicating that the subject is in a resting state in a case where the index value is a first threshold or more.

5. The blood flow analysis device according to claim 4, wherein
the correlation analysis unit outputs an estimation result indicating that the subject is in an active state in a case where the index value is less than the first threshold and a second threshold or more that is smaller than the first threshold.

6. The blood flow analysis device according to claim 5, wherein
the correlation analysis unit outputs an estimation result indicating that the subject is in a transition state in which the subject makes a transition between a resting state and an active state in a case where the index value is less than the second threshold.

7. The blood flow analysis device according to claim 3, wherein
the correlation analysis unit outputs an estimation result indicating that a motion state of the subject changes in a case where a temporal change rate of the index value is equal to or more than a third threshold, and
the correlation analysis unit outputs an estimation result indicating that a motion state of the subject is stable in a case where a temporal change rate of the index value is less than the third threshold.

8. The blood flow analysis device according to claim 1, wherein
the blood flow comparison unit compares a blood flow in a first measurement region in which a blood flow rate changes due to arteriovenous anastomosis with a blood flow in a second measurement region in which a change in the blood flow rate due to arteriovenous anastomosis is smaller than that in the first measurement region so as to estimate a change in arteriovenous anastomosis and output a result thereof.

9. The blood flow analysis device according to claim 8, wherein
the blood flow comparison unit estimates a change in the arteriovenous anastomosis to detect a change in balance of an autonomic nerve of the subject and outputs a result thereof.

10. The blood flow analysis device according to claim 1, wherein
the blood flow analysis device further includes a subject tracking unit that identifies a position of the subject before and after movement of the subject in the visible light image when the subject moves, and
the blood flow distribution detection unit detects blood flow distribution at the position identified by the subject tracking unit.

11. The blood flow analysis device according to claim 1, wherein
the blood flow analysis device further includes a face tracking unit that identifies a face portion of the subject in the visible light image, and
the blood flow distribution detection unit detects blood flow distribution in the face portion identified by the face tracking unit.

12. A biological information analysis system comprising:
the blood flow analysis device according to claim 1; and
a state estimation unit that estimates a motion performed by the subject by using a blood flow analyzed by the blood flow analysis device.

13. The biological information analysis system according to claim 12, further comprising:
an index database that describes a relationship between an index value representing a state of the blood flow and a motion performed by the subject, wherein
the state estimation unit estimates a motion performed by the subject by referring to the index database by using the index value.

14. The biological information analysis system according to claim 13, further comprising:
a correction value database in which a correction value for correcting the index value is described for each of the subjects, wherein
the state estimation unit acquires the correction value corresponding to the subject from the correction value database, and
the state estimation unit estimates a motion performed by the subject by referring to the index database by using the index value corrected by using the correction value acquired from the correction value database.

15. The biological information analysis system according to claim 13, further comprising:
a learning unit that learns a relationship between the index value and a motion performed by the subject by machine learning, wherein
the index value is a value representing a state of the blood flow, the value being acquired by the blood flow analysis device or a device other than the blood flow analysis device, and
the learning unit updates the index database according to a learned relationship.
